(19) United States
(12) Patent Application Publication
(10) Pub. No.: US 2012/0055479 A1
(43) Pub. Date: Mar. 8, 2012

(54) RESPIRATOR AND ADJUSTMENT METHOD FOR SAME

(75) Inventors: Harri Friberg, Mauren (LI); Jakob Daescher, Flaesch (CH)

(73) Assignee: INFORMATION- MANAGEMENT- TECHNOLOGY AG, Buchs (CH)

(21) Appl. No.: 13/264,001

(22) PCT Filed: May 10, 2010
(86) PCT No.: PCT/IB10/52043

§ 371 Date: Nov. 13, 2011

(30) Foreign Application Priority Data
May 28, 2009 (CH) 00817/09

(51) Int. Cl. A61M 16/00

(52) U.S. Cl. 128/204.23

(57) ABSTRACT

A respirator for respiration of a patient having an interface (3) for input of at least one respiration parameter (PINSP, PEEP, RAM, I/E, RAT, T_{INSP}, T_{EXP}, T_{AZ}) which is relevant for the respiration is provided. According to the invention, the respirator comprises a computation unit for calculating a respiration curve (4, 4a, 4b) from the at least one respiration parameter (PINSP, PEEP, RAM, I/E, RAT, T_{INSP}, T_{EXP}, T_{AZ}) and a display unit (2) for display of the calculated respiration curve (4, 4a, 4b). In addition, a method for operation of the inventive respirator is provided.

This application is a section 371 national-phase entry of PCT International application no. PCT/182010/052043 filed on May 10, 2010 and published as WO2010/136923A1 on Dec. 2, 2010; application no. PCT/182010/052043 claims benefit of priority to Swiss application no. 00817/09 filed on May 28, 2009; the entirety of PCT International application no. PCT/182010/052043 is expressly incorporated herein by reference, for all intents and purposes, as if identically set forth in full herein.

### BACKGROUND

The invention relates to a respirator for artificial respiration of a patient, comprising an interface for input of at least one respiration parameter which is relevant for the respiration, a computation unit for calculating a respiration curve from the at least one respiration parameter and a display unit for displaying the calculated respiration curve. In addition, the invention relates to a method for adjusting a respirator for the respiration of a patient, such that at least one respiration parameter that is relevant for the respiration is input. Finally, the invention also relates to a computer program product.

A respirator is an electrically or pneumatically operated machine for artificial respiration of persons whose own respiration is inadequate or has stopped. One criterion for adjusting respirators is the type of application. In invasive respiration the patient is either intubated or has had a tracheotomy. In noninvasive respiration, however, the patient is respirated through a tight-fitting mask. Another classification of respirators can be made according to emergency respirators, intensive care respirators and home respirators, on the basis of their field of use.

Essentially a distinction is made between volume-controlled, pressure-controlled and time-controlled forms of respiration. In volume control, respiration or inspiration is continued, for example, until reaching a defined inspiration volume. Accordingly, in pressure-controlled respirators inspiration is continued until reaching a preset respiratory tract pressure. Time-controlled respirators, however, operate for a preset period of time. For control of respirators, a maximum pressure or a maximum volume, for example, is input, so that when this maximum is reached, the machine switches to the expiration phase. By varying the control parameters and the respiration parameters, many different ideal respiration curves can be set and thus many respiration techniques can be utilized for respiration therapy.

Therefore different respiration parameters are used (e.g., respiration pressure, expiration pressure, the increase in pressure in inspiration, the ratio between inspiration time and expiration time, the set respiration rate, etc.), depending on the disease syndrome. To some extent, these settings are made by the operator of a respirator under great tension or under hectic conditions, for example, when there is an acute situation. The economic pressure in the medical sector has also resulted in a decline in the funds available for training operators, so that even less-trained individuals are also operating respirators. All this increases the risk of mistakes in input which, can lead to serious health consequences for the patient or in the extreme case may even result in death of the patient.

WO 02/058619, for example, also discloses a respirator which can calculate a respiration curve from certain parameters that have been input and can display it on a display device. Although this provides reasonable assistance for the operator, this respirator also presupposes a relatively great deal of technical knowledge and/or there is still a high risk of mistakes in input by operating personnel under stress.

SUMMARY

The object of the present invention is therefore to provide an improved respirator and/or an improved adjustment method, in particular a method in which the operation is simplified.

According to the invention, this object is achieved by a respirator having the features as claimed herein, namely by a respirator of the type defined in the introduction in which one or more parameters from the following group are provided as respiration parameters: ideal respiration parameters, actual respiration parameters, or reference respiration parameters; and accordingly, one or more parameters from the following group are provided as the respiration curve: ideal respiration curve, actual respiration curve, or reference respiration curve.

In addition, the invention includes a method of the type defined in the introduction, in which a respiration curve is calculated from the at least one respiration parameter, and the calculated respiration curve is displayed on a display unit.

Finally, the invention includes a computer program product with a computer program stored in it, such that it can be loaded into a memory of a respirator and executes the inventive method when the computer program is executed in the respirator.

According to the invention, this results in the operator directly receiving a visual impression of the respiration curve that has been set. In addition, the effects of altered respiration parameters are directly visible. This is an essential contribution to the state of the art because the risk of mistakes in input, and thus the risk of health impairment for the patient, can be greatly reduced.

In particular, ideal respiration parameters and an ideal respiration curve represent a desired respiration characteristic set on the machine, while actual respiration parameters and an actual respiration curve represent a respiration characteristic actually determined on a patient. Reference respiration parameters and a reference respiration curve represent an optimal respiration characteristic on a “normal patient.” For example, the inspiration pressure, the expiration pressure, the increase in pressure during inspiration, the ratio between inspiration time and expiration time and the respiration rate, for example, are also taken into account as respiration parameters. In comparison with known approaches, the important advantage is therefore that different types of respiration curves can be displayed. In other words, the respirator does not merely reflect the information input by the operator, even if it is in graphic form, but instead may also display an actual respiration characteristic that has been determined and a stored or determined reference respiration characteristic in addition to the set ideal respiration characteristic. The reference respiration characteristic in particular provides valuable assistance for a less trained, uncertain, or stressed operator.

The computation unit in the respirator may be implemented in software and/or hardware. It is especially advantageous if a program which images the inventive method among other things and is stored in a memory is executed by a processor. The algorithm can be adapted especially easily to different conditions in this way.

Advantageous embodiments and refinements of the invention are derived from the dependent claims as well as the description in conjunction with the drawings in the figures.

It is advantageous if the interface is provided for input of the at least one respiration parameter by evaluation of a drag-and-drop procedure performed by an operator on the respiration curve displayed on the screen. This allows a particularly intuitive adjustment of a respiration curve which can be performed reliably by an operator of the respirator even under high-stress conditions. A respiration curve in a certain range is selected and then modified as desired by dragging it. It is particularly advantageous if a touchscreen is used because the curve can then be adjusted easily using a finger.

It is also advantageous if one or more of the following group of at least one patient parameter characterizing the patient is determined: ideal respiration parameter, ideal respiration curve, reference respiration parameter or reference respiration curve. As mentioned above, the reference respiration parameter and a reference respiration curve represent an optimal respiration behavior on a normal patient. This optimal respiration characteristic is of course different from one patient to the next and depends on certain patient parameters. For example, age, weight, gender, general fitness and disease status may influence the aforementioned optimal respiration behavior. According to the invention, optimal reference respiration parameters or an optimal reference respiration curve can be determined from at least one patient parameter and may be offered to the operator of the respiration device as assistance. In an especially advantageous embodiment, ideal respiration parameters or an ideal respiration curve can be determined from the at least one patient parameter, so that the adjustment work is reduced to a minimum. For example, a key with which the reference respiration parameter is assigned to the ideal respiration parameters or the reference respiration curve is assigned to the ideal respiration curve may also be provided.

It is advantageous if the respirator comprises means for measuring the actual respiration curve and/or the actual respiration parameters at an output of a respiration tube. The actual respiration characteristic prevailing with the patient can be ascertained in this way and then used as a basis for deriving additional treatment measures.

It is also advantageous if the respirator comprises means for measuring the actual respiration curve and for determining an actual respiration parameter from the actual respiration curve determined. Alternatively, it is also advantageous if the respirator comprises means for measuring an actual respiration parameter and for determining the actual respiration curve from the actual respiration parameter determined. In such variants, the actual respiration parameters are determined from a measured actual respiration curve, (i.e., the pressure or volume curve over time). For example, the inspiration pressure can then be determined very easily by forming the maximum value. However, the opposite path is also possible.

For example, an actual respiration curve can be calculated from measured actual respiration parameters. For example, the inspiration pressure, the expiration pressure, the inspiration time and the expiration time are measured for this purpose. In principle, the same algorithm which is also used for calculating the ideal respiration curve or the reference respiration curve may be used for this. However, the actual respiration parameters are input by using a measurement apparatus, whereas the ideal respiration parameters are input by using a keypad on the respirator, for example. However, the reference respiration parameters can be input via a table stored in the respirator or in a stored database.

It is also advantageous if the display unit is prepared for displaying at least one respiration parameter on the display unit. This is further support for the operator of the respirator, which also has a training effect. By displaying the respiration parameters, these parameters are also impressed upon the operator—even unconsciously—so that operation of the respirator can be accomplished more rapidly and reliably over time. Multiple respiration parameters may of course also be placed side by side. For example, the actual inspiration pressure may be displayed next to the ideal inspiration pressure, so that deviations between the two values are visible immediately.

In an advantageous variant of the invention the respirator comprises means for output of a warning when the difference and/or the ratio between the two different respiration parameters exceeds a preselectable value. This monitors automatically whether the deviation between the actual inspiration pressure addressed above, for example, and the ideal inspiration pressure exceeds a predetermined threshold value. If this is the case, a visual warning and/or an acoustic warning is then output to draw the attention of the operator to this circumstance. The threshold value may be input by the operator himself or may be provided at the factory.

In another advantageous variant of the present invention, the respirator comprises means for determining the area between two different respiration curves during an observation period and for output of a warning when the area exceeds a predetermined value. This is another possibility for checking on whether the respiration is taking place in the desired manner or whether the ideal values that have been input are plausible with respect to the prevailing reference values. Not only are individual respiration parameters, for example, the expiration pressure, monitored here, but also the course of the respiration curve is monitored.

It is advantageous if the observation period is longer than the duration of one breath. For monitoring a respiration parameter and also for monitoring a respiration curve, an observation period which is longer than the duration of one breath is advantageously chosen. In this way, individual disturbances in the respiration process, e.g., coughing by the patient or individual measurement errors can be suppressed better. The observation period may be determined on the basis of a time indication or also on the basis of a number of breaths (e.g., 2.7 breaths).

Finally, it is advantageous if the respirator comprises a memory for permanent storage of a set of respiration parameters and/or a respiration curve. In this way, the respiration parameters are preserved, for example, even after the respirator has been shut down and can thus be retrieved conveniently, for example, when the same patient must be respirated repeatedly with pauses in between as part of a respiration therapy.

In conclusion, it is pointed out that the variants mentioned with respect to the inventive respirator and the resulting advantages are not based only on the respirator itself but also on the inventive method. Those skilled in the art will easily be able to adapt the teaching disclosed here to the inventive method.

The above embodiments and refinements of the invention can be combined in any desired manner.

### BRIEF DESCRIPTION OF THE DRAWINGS

The present invention will be explained in greater detail below on the basis of the exemplary embodiments shown in the schematic drawings in the figures, in which:

FIG. 1 shows a schematic operating field of an exemplary respirator;

FIG. 2 shows an arrangement in which two respiration curves are shown instead of the respiration curve from FIG. 1;

FIG. 3 shows the area between two respiration curves;

FIG. 4 shows how one respiration curve can be modified with the help of a drag-and-drop procedure.

### DETAILED DESCRIPTION

The same and similar parts are labeled with the same reference numerals in the drawings in the figures, and elements and features having a similar function are provided with the same reference numerals but with different indices—unless otherwise indicated.

FIG. 1 shows a schematic operating field 1 of an exemplary respirator including a display unit 2 and several operating keys 3. A respiration curve 4 is shown on the display unit 2. In addition, an area 5 in which respiration parameters are displayed is shown on the display unit 2.

In a first example, ideal respiration parameters are input by the operator of the respirator via the operating keys 3 of the respirator. The following parameters are fixedly assigned to the operating keys in this example: inspiration pressure PINSP, expiration pressure PEEP, the increase in pressure in inspiration RAM, the ratio between inspiration time and expiration time I/E and the respiration rate RAT. FIG. 1 also shows the importance of the inspiration pressure PINSP, the expiration pressure PEEP, the increase in the pressure in inspiration RAM, the inspiration time T_{INSP} and the expiration time T_{EXP} (note: this information is not necessarily also displayed on a display unit 2 and serves here primarily to provide a better understanding of the invention). The ratio between the inspiration time T_{INSP} and the expiration time T_{EXP} now corresponds to I/E, the sum of the two times 1/respiration rate RAT.

According to the prior art, an operator must know in detail the effects of the individual parameters on respiration, which often leads to mistakes in operation, especially with the stress associated with emergency situations. The inventive method then calculates an ideal respiration curve 4 for the input parameters and displays this on the display unit 2. This is an enormous assistance for the operator because the effect of a certain input is shown visually immediately. In an advantageous variant of the invention the parameters themselves are also displayed in an area 5. The operator can thus become more easily familiar with such conventional parameter sets and can adjust the respirator more rapidly the next time accordingly.

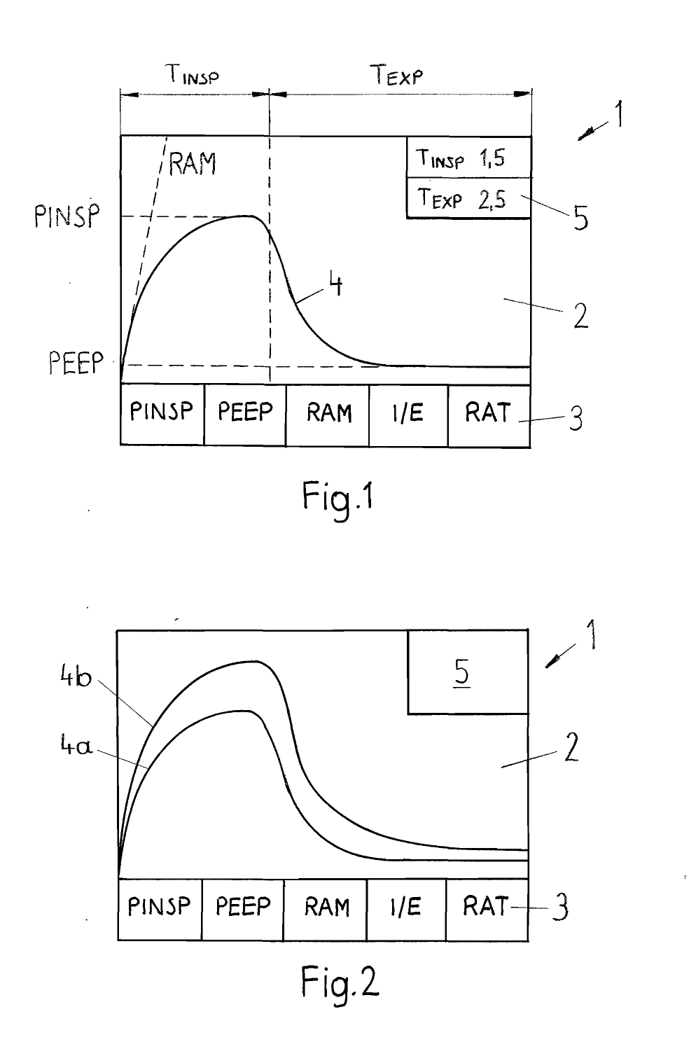

FIG. 2 shows an arrangement which is very similar to the arrangement in FIG. 1. However, instead of the respiration curve 4, two respiration curves 4a and 4b are shown here.

In a first example, the respiration curve 4a is a reference respiration curve, while respiration curve 4b is an ideal respiration curve. In this example, the operator of the respirator inputs patient parameters, which characterize the patient. These may include, for example, weight, age, gender, severity of illness, etc. The respirator then calculates a reference respiration curve 4a from the patient parameters and displays this on the display unit 2. In the next step the operator inputs ideal respiration parameters for the patient. The respirator calculates an ideal respiration curve 4b from these parameters as already mentioned above and also displays this on the display unit 2. Then the operator sees not only the effects of the various ideal respiration parameters on the basis of the ideal respiration curve 4b but also a deviation from the reference respiration curve 4a. In this way, the operator can “approach” an ideal reference respiration curve 4a or may also deviate from it intentionally because, for example, there are medical reasons against the use of a reference respiration curve. In an advantageous variant, the reference respiration parameters may be assigned automatically to the ideal respiration parameters and/or the reference respiration curve may be assigned automatically to the ideal respiration curve, so that the adjustment work is facilitated. This step may also be performed automatically after input of the patient parameters.

In a second example, the respiration curve 4a is an actual respiration curve while the respiration curve 4b is in turn an ideal respiration curve. In this constellation, the operator of the respirator can check on the extent to which the actual respiration curve 4a measured on the patient corresponds to the set ideal respiration curve 4b and may initiate countermeasures accordingly as needed, i.e., when there is a great deviation.

In a third example, the respiration curve 4a is an actual respiration curve but the respiration curve 4b is a reference respiration curve. In this variant, the operator of the respirator may check on the extent to which the actual respiration curve 4a measured on the patients corresponds to a reference respiration curve 4b, which is calculated on the basis of at least one patient parameter characterizing the patient. In this way, the operator can ascertain the extent to which the actual respiration curve 4a deviates from the “norm.”

In the examples given above, actual respiration curve is always measured, i.e., a curve of pressure over time was measured. However, a variant in which the actual respiration parameters determined are merely input, i.e., measured, and an actual respiration curve is calculated from them is also conceivable. In this case, for example, the inspiration pressure (i.e., the maximum pressure during the respiration process), the expiration pressure PEEP (i.e., the minimum pressure during the respiration process), the inspiration time (time between the increase in pressure and the drop in pressure) and the expiration time (time between the pressure drop and the pressure rise) may be measured and an actual respiration curve calculated from them. However, actual respiration parameters may of course also be determined from the actual respiration curve. For example, the inspiration pressure PINSP can be determined by evaluating the actual respiration curve (i.e., determining its maximum value).

In another advantageous embodiment of the invention, various respiration parameters can be compared with one another in the area 5 of the display unit 2. For example, the reference respiration parameters may be compared with the ideal respiration parameters (Example 1), the actual respiration parameters may be compared with the ideal respiration parameters (Example 2) or the actual respiration parameters may be compared with the reference respiration parameters (Example 3). In the upper right corner of the display unit, a corresponding table is advantageously inserted (note: however, no concrete values are shown in area 5 in this example).

In an advantageous variant of the invention, an ideal respiration curve, a reference respiration curve and an actual respiration curve and/or ideal respiration parameters, reference respiration parameters and actual respiration parameters may of course also be displayed at the same time.

In another advantageous variant of the invention, a warning may be output when the difference, i.e., the ratio between two different respiration parameters exceeds a predetermined value.

For example, a deviation of the actual inspiration pressure from the ideal inspiration pressure or a deviation in the ideal respiration rate from the reference respiration rate may thus be ascertained and the operator of the respirator may be thereby notified. Although an optical display is sufficient in the case of a deviation in a ideal respiration parameter from a reference respiration parameter (it is assumed that the operator has turned his/her attention to the display unit 2 while setting the respirator), preference should be given to an (additional) acoustic warning in the case of a deviation of an actual respiration parameter from an ideal or reference respiration parameter.

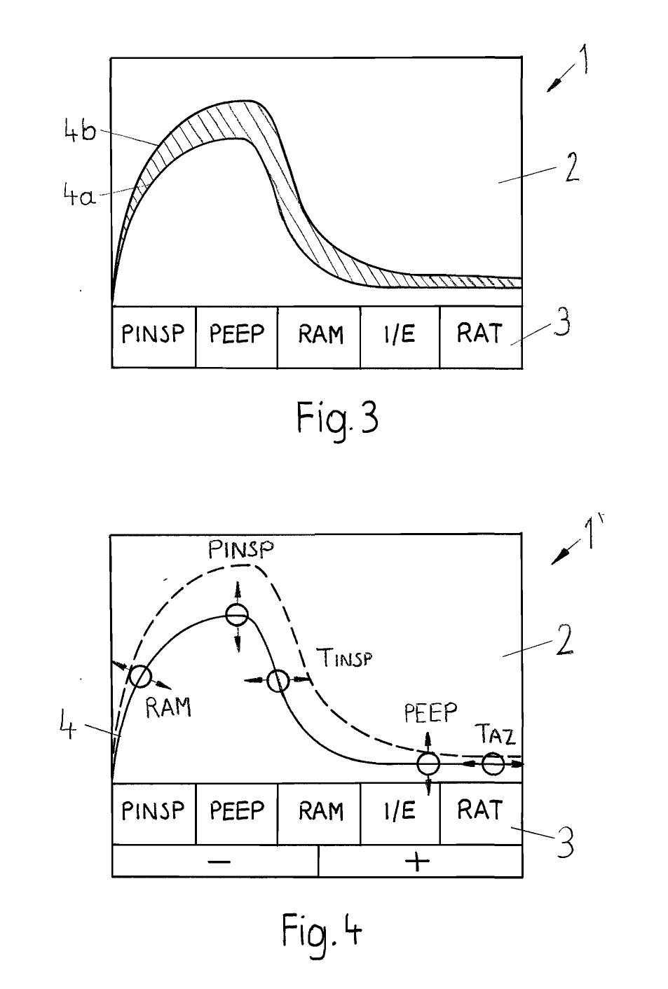

Similarly, the area between two different respiration curves may be used for output of a warning during an observation period. For example, the area between the actual respiration curve and the ideal respiration curve or between the ideal respiration curve and the reference respiration curve may be determined and a warning may be output when the difference exceeds a certain measure (in this regard, see the hatched area in FIG. 3).

For monitoring a respiration parameter and also for monitoring a respiration curve, an observation period which is longer than the duration of one breath is advantageously used. In this way, individual disturbances in the respiration process, e.g., coughing by the patient or individual measurement errors, can be suppressed better. The observation period may be determined on the basis of a time specification or on the basis of a number of breaths (e.g., 2.7 breaths).

In another variant respiration parameters and/or respiration curves and/or patient parameters may also be stored permanently. These are then preserved, for example, even after the respirator has been turned off and can thus be retrieved again conveniently, for example, if the same patient must be respirated repeatedly with pauses in between.

In the previous examples and variants, it has been assumed that the respiration parameters can be set by means of keys 3, buttons, regulators and the like provided on the machine. In an advantageous variant, these input elements are arranged on a touchscreen. So-called “soft keys” may be implemented in this way; these are adjustment elements which can change the function assigned to them. For example, the same keys may be provided for input of respiration parameters or patient parameters, depending on the set function.

In a particularly advantageous variant, the respiration curve may be modified directly, i.e., without a change in a certain respiration parameter. To do so a respiration curve and/or an area of same is selected on the touchscreen and modified by dragging it. This method is best known by the term “drag-and-drop.” Operation of a respirator thus becomes more intuitive. FIG. 4 shows examples of points on a respiration curve 4, where the curve can be “captured” by using a finger and then dragged. The arrows symbolize the direction in which dragging is possible, for example, producing a change in the respiration curve 4. The values of the respective respiration parameters are of course continuously adapted to the new respiration curve 4 and displayed. Use of a drag-and-drop procedure is of course not limited to use of a touchscreen but may also be performed in conjunction with other input devices, for example, in combination with keys, levers or a computer mouse. The arrangement of the areas where the respiration curve 4 can be captured are also to be seen as mere examples. In particular for the change in the ratio between the inspiration time and the expiration time I/E and the respiration rate RAT and/or respiration time T_{AZ}, areas may also be provided at the side of the respiration curve 4, for example, at the upper or lower edges of the display screen.

FIG. 4 also shows additional plus and minus keys with the help of which the value of a previously selected respiration parameter can be altered (for example, with the keys 3 from FIGS. 1 to 3, the value may be increased by pressing in the upper/right area of the key 3 or the value may be lowered by pressing in the lower/left area of the key 3).

In conclusion, it is pointed out that the various exemplary embodiments depicted in the figures may also form the basis for independent inventions.

In addition, it is noted that the invention of course refers not only to pressure-controlled respirators but also to volume-controlled or time-controlled respirators, although this is not depicted explicitly in the figures. Those skilled in the art will be able to easily apply the teaching disclosed here to the aforementioned fields, in particular by employing different respiration parameters, e.g., the inspiration volume.

### List of Reference Numerals

1, 1′ operating field
2 display unit
operating keys
4, 4a, 4b respiration curve
5 area for respiration parameters
I/E ratio between inspiration time and expiration time
PEEP expiration pressure
PINSP inspiration pressure
RAM increase in pressure in inspiration
RAT respiration rate
T_{AZ} respiration time T_{EXP} expiration time
T_{INSP} inspiration time

1-22. (canceled)

23. A respirator comprising:
an interface configured to receive input of at least one respiration parameter selected from at least one of the group of respiration parameters consisting of PINSP, PEEP, RAM, I/E, RAT, T_{INSP}, T_{EXP}, and T_{AZ},
a computation unit configured to calculate, from said at least one respiration parameter, a first respiration curve selected from the group of respiration curves consisting of ideal respiration curve, actual respiration curve, and reference respiration curve, said computation unit being in operative communication with said interface;
said computation unit calculating a second respiration curve selected from the group of respiration curves consisting of ideal respiration curve and reference respiration curve, wherein said second respiration curve is not identical to said first respiration curve;
said computation unit determining the area between said first respiration curve and said second respiration curve over a selected observation period, said computation unit signaling a warning when said area exceeds a predetermined value; and,
a display unit configured to display at least one of said first and second respiration curves, said display unit being in operative communication with said computation unit.

24. . The respirator as claimed in claim 23, wherein:
said interface is a drag-and-drop interface; and,
said computation unit determines, from at least one characterizing patient parameter, at least one patient parameter from at least one of the group consisting of ideal respiration parameter, ideal respiration pressure curve, reference respiration parameter, and reference respiration pressure curve.

25. A respirator as claimed in claim 23, further comprising:
at least one sensor for measuring the actual respiration pressure curve.

26. A respirator as claimed in claim 23, further comprising:
at least one sensor for measuring an actual respiration pressure parameter.

27. A respirator as claimed in claim 23, further comprising one of:
at least one sensor for measuring the actual respiration pressure curve as a determinant of at least one actual respiration parameter; or,
at least one sensor for measuring at least one actual respiration parameter as a determinant of the actual respiration pressure curve.

28. The respirator as claimed in claim 23, wherein:
said display unit is configured to display at least one respiration parameter.

29. The respirator as claimed in claim 23, wherein:
said computation unit signals a warning when the difference between two different respiration parameters exceeds a predetermined value.

30. The respirator as claimed in claim 23, wherein:
said computation unit signals a warning when the ratio between two different respiration parameters exceeds a predetermined value.

31. A respirator as claimed in claim 23, further comprising:
a memory apparatus configured to permanently store respiration data selected from the group consisting of a set of respiration parameters, a respiration pressure curve, and a set of respiration parameters with a respiration pressure curve.

32. A method for adjusting a respirator comprising the steps of:
inputting at least one respiration parameter into an interface;
calculating a first respiration curve from the at least one respiration parameter;
establishing a second respiration pressure curve;
displaying the first respiration pressure curve on a display;
determining the area between the first and second respiration pressure curves during an observation period; and,
outputting a warning when the area exceeds a predetermined value.

33. A method for adjusting a respirator as claimed in claim 32, further comprising the steps of:
evaluating a drag-and-drop on the first respiration pressure curve displayed on the display; and,
inputting at least one respiration parameter based on said step of evaluating.

34. . A method for adjusting a respirator as claimed in claim 32, further comprising the steps of:
providing at least one respiration parameter selected from at least one of the group of respiration parameters consisting of PINSP, PEEP, RAM, I/E, RAT, T_{INSP}, T_{EXP}, and T_{AZ}; and,
providing at least one respiration pressure curve selected from at least one of the group consisting of ideal respiration pressure curve, actual respiration pressure curve, and reference respiration pressure curve.

35. A method for adjusting a respirator as claimed in claim 34, further comprising the step of:
determining at least one patient parameter selected from at least one of the group consisting of ideal respiration parameter, ideal respiration pressure curve, reference breathing parameter, and reference respiration pressure curve.

36. A method for adjusting a respirator as claimed in claim 35, further comprising the step of:
measuring at an output of a respirator tube the actual respiration pressure curve, or an actual respiration pressure parameter, or both the actual respiration pressure curve and an actual respiration pressure parameter.

37. A method for adjusting a respirator as claimed in claim 36, further comprising the steps of:
measuring the actual respiration pressure curve; and,
determining the actual respiration parameter from the measured actual respiration pressure curve.

38. A method for adjusting a respirator as claimed in claim 36, further comprising the steps of:
measuring the actual respiration parameter; and,
determining the actual respiration pressure parameter from the measured actual respiration parameter.

39. A method for adjusting a respirator as claimed in claim 32, further comprising the step of:
displaying at least one respiration parameter on the display.

40. A method for adjusting a respirator as claimed in claim 32, further comprising the step of:
outputting a warning when the difference between two different respiration parameters exceeds a predetermined value.

41. A method for adjusting a respirator as claimed in claim 32, further comprising the step of:
outputting a warning when the ratio between two different respiration parameters exceeds a predetermined value.

42. A method for adjusting a respirator as claimed in claim 32, further comprising the step of:
permanently storing respiration data including a set of respiration parameters, or at least one respiration pressure curve, or a set of respiration parameters with at least one respiration pressure curve.
